(19) United States
(12) Patent Application Publication
(10) Pub. No.: US 2016/0303035 A1
(43) Pub. Date: Oct. 20, 2016

(54) Toothpaste Composition Containing Curcuma Longa L. and Method of Manufacturing Same

(71) Applicant: BIO NANO TRINITY. CO. LTD, Gunsan-si, Jeollabuk-do (KR)

(72) Inventor: Jin-ok KIM, Gunsan-si (KR)

(21) Appl. No.: 15/102,197

(22) PCT Filed: Jun. 30, 2015
(86) PCT No.: PCT/KR2015/006669

§ 371 Date: Jun. 6, 2016

(30) Foreign Application Priority Data
Jul. 16, 2014 (KR) 10-2014-0089430

(51) Int. Cl. A61K 8/97; A61Q 11/00; A61K 8/34; A61K 8/368; A61K 8/19; A61K 8/26; A61K 8/98; A61K 8/23; A61K 8/73

(52) U.S. Cl. CPC A61K 8/97; A61K 8/23; A61Q 11/00; A61K 8/345; A61K 8/73; A61K 8/19; A61K 8/26; A61K 8/988; A61K 8/368; A61K 2800/28; A61K 2800/74; A61K 2800/85

(57) ABSTRACT

The present invention aims to provide a highly functional toothpaste composition that can achieve the effects of not only preventing tooth decay, but also removing halitosis, removing inflammation, alleviating oral diseases, treating sensitive teeth, and whitening teeth using natural materials.

To this end, the present invention comprises: Curcuma longa L.; natural organic sulfur; mint extract; red ginseng fermentation extract; and antibacterial combined extract made by extracting at least one from the group consisting of green tea, dried orange peel, Houttuynia cordata Thunb., Rumex crispus, licorice, and Mori Cortex Radicis.

### TECHNICAL FIELD

The present invention relates to a toothpaste composition containing Curcuma longa L. and a manufacturing method thereof, and more specifically, to a highly functional toothpaste composition and a manufacturing method thereof, the composition that which uses natural materials to not only prevent tooth decay but also remove halitosis and inflammation, improve oral diseases, treat sensitive teeth, and whiten the teeth.

### BACKGROUND

Recently, active efforts are being made to achieve the effects of periodontal diseases or halitosis removal, sensitive teeth prevention, and teeth whitening through functional toothpastes.

As a prior art, a toothpaste composition containing Geumgang pine extract and a manufacturing method of the toothpaste of Korea patent registration No. 10-0859967 discloses a toothpaste composition manufactured by pre-treating (extract, refine, concentrate, etc.) a Geumgang pine, the toothpaste composition thereby containing phytoncide and having antibacterial and antiseptic effects.

Also, a toothpaste composition containing Rumex crispus extract as an active ingredient of Korea patent registration No. 10-0999406 discloses a toothpaste composition using Rumex crispus extract for suppressing and sterilizing germs that become causes of gum diseases, alleviating and treating inflammation to alleviate, treat, and prevent oral inflammation or gum diseases.

However, the toothpaste composition having Geumgang pine extract and the manufacturing method thereof from the prior art have limitations in mass production because of the difficulty in obtaining raw materials due to limited availability of raw materials among other reasons and has technical challenges due to the complexity in the raw material processing technology.

Also, the toothpaste composition containing the Rumex crispus extract as an active ingredient of the prior art uses the Rumex crispus extract and Houttuynia cordata Thunb. powder as main ingredients of the toothpaste but using said ingredients alone does not lead to satisfactory effects of removing periodontal diseases or halitosis, preventing sensitive teeth, or teeth whitening. Therefore, there is a pressing need for a development of a toothpaste component that contains natural materials as active ingredients so as to enable easy procurement, and that which allows for combined effects of not only tooth decay prevention but also periodontal diseases or halitosis removal, sensitive teeth prevention, and teeth whitening.

### DETAILED DESCRIPTION OF THE INVENTION

### Technical Problem

The present invention was thought out to solve said problem, and the present invention aims to provide a highly functional toothpaste composition and a manufacturing method thereof, the toothpaste composition which uses natural materials to achieve the effects of not only preventing tooth decay but also removing halitosis, removing inflammation, improving oral diseases, treating sensitive teeth, and whitening teeth.

### Means for Solving the Problem

In order to achieve said purpose, the toothpaste composition containing Curcuma longa L. of the present invention comprises Curcuma longa L. powder, natural organic sulfur, mint extract, red ginseng fermentation extract, and antibacterial combined extract made by extracting at least one from the group consisting of green tea, dried orange peel, Houttuynia cordata Thunb., Rumex crispus, licorice, and Mori Cortex Radicis.

In a preferred embodiment of the toothpaste composition of the present invention, the toothpaste composition comprises 1 to 5 wt % of the Curcuma longa L. powder, 0.5 to 1.5 wt % of the natural organic sulfur, 1 to 3 wt % of the mint extract, 0.5 to 2.5 wt % of the red ginseng fermentation extract, and 7 to 10 wt % of the antibacterial combined extract to the total composition.

Also, the toothpaste composition of the present invention can further comprise xylitol, vegetable glycerin, xanthan gum, Yucca recurvifolia extract, sodium cocoyl apple amino acid, sodium phytate, calcium carbonate or bicarbonate, kaolin clay, bamboo salt, propolis, natural anisic acid antiseptic having star anise as an ingredient, lemon essential oil, peppermint essential oil, and spearmint essential oil.

In another embodiment of the present invention, it is preferred for the calcium carbonate that can be included in the toothpaste composition to be made into nano-sized calcium carbonate.

A preferred embodiment of the manufacturing method of the toothpaste composition containing Curcuma longa L. of the present invention comprises the steps of:
a) Placing and heating up the vegetable glycerin in container A, and then placing and dissolving the xylitol therein, and then placing and dissolving the xanthan gum therein;
b) Mixing in container B the mint extract, the red ginseng fermentation extract, and the antibacterial combined extract made by extracting at least one from the group consisting of the green tea, the dried orange peel, the Houttuynia cordata Thunb., the Rumex crispus, the licorice, and the Mori Cortex Radicis, and then adding thereto the Yucca recurvifolia extract, the sodium cocoyl apple amino acid, and the sodium phytate, and then mixing therewith the composition obtained in the step a);
c) Adding and mixing the calcium carbonate, the kaolin clay, the Curcuma longa L. powder, the natural organic sulfur, and the bamboo salt respectively to the composition obtained in the step b); and
d) Adding the propolis, the anisic acid natural antiseptic having star anise as an ingredient, the lemon essential oil, the peppermint essential oil, and the spearmint essential oil to the composition obtained in the step c).

### Effect of the Invention

The toothpaste composition of the present invention can be prepared using natural materials as an active ingredient and is thus easy to obtain and is highly stable for applying to the human body.

Also, the present invention comprises the Curcuma longa L. powder, the natural organic sulfur, and the antibacterial combined natural extracts enabling not only very effective prevention of tooth decay and gum diseases (bleeding gums) through excellent antibacterial and anti-inflammatory functions, but also treatment of sensitive teeth and teeth whitening (removal of plaque) effects.

### SIMPLE DESCRIPTION OF DRAWING

FIG. 1 is a drawing that roughly describes the ingredients and the manufacturing process of the toothpaste composition of the present invention.

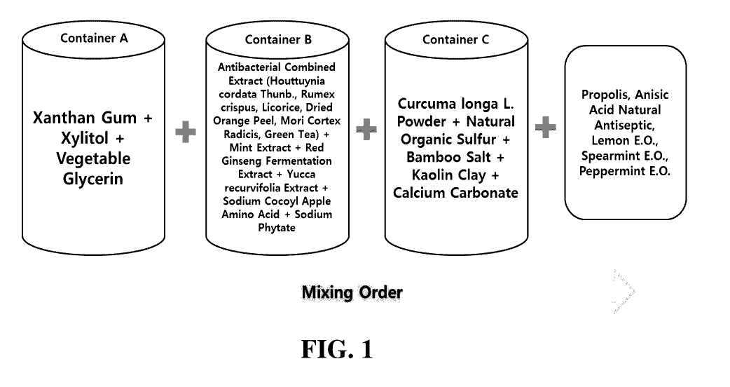

### BEST MODE FOR CARRYING OUT THE INVENTION

The Curcuma longa L. used in the toothpaste composition of the present invention is a perennial herb plant belonging to the Zingiberaceae group, similar in appearance to turmeric but characterized by both sides of the leaf being smooth. It is grown naturally and cultivated in Southeast Asian regions including southern China, India, and Okinawa and also cultivated in central and southern regions of Korea.

The Curcuma longa L., according to recent research in several countries, has been known for yellow color Curcumin, which is the main component of the Curcuma longa L. and the substance that carries out anti-oxidation and cell protection (Chatcrjrr et al., 1999) and the anti-oxidation activity is reported to protect numerous peroxidation related diseases in the body (Prriyadarsini, 1997). As such, many reports are actively in progress on the Curcumin and several papers have been published. Some of the most well-known papers introduce anti-oxidation (Kang et al., 1998; Selvam et al., 1995; Jeong et al., 2004), anticancer (Ryu et al., 2005; Khar et al., 1995), antiviral (Mazumder et al., 1995), and anti-inflammatory (Ammon & Wahl, 1991; Gupta & Gahsh, 1999) functions and in addition, the Curcuma longa L. has been introduced in several papers for dropping blood lipid levels and having anti-inflammatory effects, inhibiting cancer cell growth related progress in blood vessels, lowering cholesterol accumulated in the liver resulting in positive effects on the liver and the nervous system. In particular, the Curcuma longa L. is known to be effective for gum diseases.

The Curcuma longa L. is the dried version of the tuberous root of the Curcuma longa L. that is in the Zingiberaceae group and the turmeric is the rootstock thereof. The turmeric belongs to same root area but in a strict sense, is different from the Curcuma longa L. Thus, the two are similar but distinctly different types of a crop. The two are both roots but is divided by the different areas. The Curcuma longa L. is closer to gray color and the turmeric is yellow colored and is used for curry.

A preferred embodiment comprises powder type Curcuma longa L. in the toothpaste composition of the present invention, which is readily available on the market.

Said natural organic sulfur (MSM, Methyl Sulfonyl Methane) in the toothpaste composition of the present invention is non-toxic non-fragrant sulfur extracted from a plant, and is 99.9% Dimethyl Sulfone water-soluble white-colored powder.

The natural organic sulfur acts as a natural antidote, natural anti-cancer agent, and natural emulsifier and gets rid of various toxins from chemicals, pollution, and heavy metals. The natural organic sulfur also protects the body and the scalp from environmental factors (hair dye, perm, strong cleansing) in addition to having the antidote effects.

Also, the sulfur is excellent at removing waste or toxins and reviving cells, good for alleviating pain and inflammation. In particular, vegetable sulfur is a non-toxin ingredient with strong properties of transferring a substance, shows medicinal properties by deeply penetrating the cell membrane of the skin and the body, and has strong effects of expanding the blood vessel and increasing the blood flow. The sulfur changes the cross-linking of collagen, which makes up connective tissues, thereby rapidly reducing the scar tissues. In particular, the sulfur works as natural antibiotics when used on teeth. Such natural organic sulfur can be obtained from publicly available products.

The mint extract contained in the toothpaste composition of the present invention is an extract of Mentha arvensis L. component, the mint being a dicotyledonous gamopetalous Lamiaceae perennial herb. The main component of the mint includes menthol, menthone, iso-menthone, camphene, and limonene.

The mint has antipyretic, anti-inflammatory, and stomach strengthening effects, induces bile secretion, mucus secretion in the respiratory tract increases, expansion of capillaries, and stimulation of the central nervous system, increases uterine contractions, and relieves boils. The mint can be used for indigestion, sore throat, tonsillitis, skin itching, abdominal pain, diarrhea, vomiting, headaches, toothaches, colds, removal of boils, and relieving bloodshot eyes.

In one embodiment of a mint extraction method of the present invention, 1 kg of the mint is pre-heated for 3 hours at 50° C. and then heated for 3 hours and 30 minutes in the range of 95 to 105° C., followed by rapid cooling and then ultrasonic low temperature extraction at 95° C.

In red ginseng fermentation extract contained in the toothpaste composition of the present invention, saponin of ginseng is known to promote the release of catecholamine in the adrenal glands, have antioxidant activity by inhibiting the peroxidation process of lipids, which in turn, inhibits the radical formation having a beneficial effect on the body, particularly effective in recovering skin function.

As a method of extracting the red ginseng extract of the present invention, the following method can be adopted as a preferred embodiment.

—Method 1—

400 g of ginseng (dried ginseng) and 400 g of fine roots of ginseng are submerged in 12 L of purified water and pre-heated for 4 hours at 50° C. and then, heated for 40 hours at 90° C., followed by 32 hours of heating at 105° C. to obtain the red ginseng extract, which is then fermented using lactic acid bacteria for 72 hours, maintaining the temperature at 37° C. Then, the extract is heated to 100° C., sterilized, and then extracted to finally obtain the red ginseng fermentation extract.

—Method 2—

Another method for fermentation is to maintain the red ginseng extract at 37 degrees by protecting from light and external temperature for 72 hours after putting in 2 g of EM pure powder per 1 L and 20 g of natural betaine powder, covered with lagging for 7 days when placing at room temperature.

The antibacterial combined extract contained in the toothpaste composition of the present invention is made by extracting and mixing at least one from the group consisting of the green tea, the dried orange peel, the Houttuynia cordata Thunb., the Rumex crispus, the licorice, and the Mori Cortex Radicis, and the efficacy can be maximized when all six of the materials are contained.

Effects of each component constituting the antibacterial combined extract of the present invention are as follows:
1) Catechin which is a polyphenol compound contained in abundance in the green tea has an effect of aging management, whitening, skin soothing and clearing, and eliminates oral halitosis.
2) The dried orange peel refers to ripe fruit skin of Rutaceae tangerine (Citrus unshiu Markovich) or closely related congeneric plants, which are high in vitamins A and C content and have an adequate amount of sugar and organic acid content. Other effects include expansion of blood vessels, and excellent anti-ulcer and diuretic effect. Yet other effects include increase in appetite and good circulation of energy, relieving thirst, stopping nausea and coughing. High content of hesperidin, which is a flavonoids compound having effects of vitamin P reducing permeability of blood vessels and increasing resistance of blood vessels thereby preventing the rupture of blood vessels, can be found in the dried orange peels.
3) The Houttuynia cordata Thunb. has an excellent anti-inflammatory effect for eliminating inflammations. In particular, the component called Quercitrin removes the toxins in the skin and has excellent blood-cleansing effects by expanding the capillary and thinning the blood.
4) The roots of the Rumex crispus reduce fever, have antibacterial effects, and treat hair loss and bruises. Other effects include excellent sterilizing effects, treating skin inflammation as well as internal inflammation of the organs. Yet other effects include inhibiting cancer cell generation and preventing metastasis, excellent antibacterial effects for gum inflammation and bleeding gums, effective for relieving swollen gums and flares.
5) The licorice allows for the recovery of the liver function by bonding with the toxic substances and having a detoxifying effect, and is effective for drug addiction, hepatitis, hives, dermatitis, and eczema, among others. Other effects include stopping cough and discharging phlegm, and acting as antihistamine and anti-acetylcholine. Yet other effects include relieving pains caused by abrupt tension in the muscle or the tissue, increase in weight and leukocyte, diuretic and anti-inflammatory effects, and in particular components such as liquiritin and liquiritingenin suppress the occurrence of peptic ulcers.
6) The Mori Cortex Radicis is extracted from mulberry rhizodermis, and can lower blood pressure, which is effective for high blood pressure and diabetes. Other effects include stopping coughs, acting as a diuretic, having soothing effects, relieving pain, dropping body temperature, antispasmodic action, and also suppressing growth of Staphylococcus aureus, Haemophilus influenzae, and dysentery bacillus. Yet other effects include excellent whitening effect.

The antibacterial combined extract of the present invention can be obtained using the manufacturing method from the following embodiment:
1) 300 g of the green tea, 300 g of the dried orange peel, and 300 g of the Houttuynia cordata Thunb. are submerged in 1500 g of 35° C. alcohol for about 3 hours, then heated for 1 hour and 30 minutes at 95° C., into which 6 L of purified water is then poured in and temperature set at 105° C., from which point 2 L of extract is extracted using low temperature vacuum extractor for 2 hours.
2) 500 g of the Mori Cortex Radicis, 600 g of the Rumex crispus, 400 g of the licorice are submerged in 1500 g of 35° C. alcohol for about 3 hours, then heated for 2 hours and 30 minutes at 105° C., into which 9 L of purified water is then poured in and temperature set at 105° C., from which point 3 L of extract is extracted using low temperature vacuum extractor for 2 hours and 30 minutes.

The extraction method may be changed by a person skilled in the art according to the type and amount of the materials contained in the extract.

The toothpaste composition containing Curcuma longa L. of the present invention may further comprise xylitol, vegetable glycerin, xanthan gum, Yucca recurvifolia extract, sodium cocoyl apple amino acids, sodium phytate, calcium carbonate or bicarbonate, kaolin clay, bamboo salt, propolis, anisic acid natural antiseptic having star anise as ingredient, lemon essential oil, peppermint essential oil, and spearmint essential oil.

Said xylitol is a sugar alcohol type natural sweetener used in chewing gum or confectionery, pharmaceutical, oral care agent or the like. The xylitol is effective in preventing tooth decay because of the pentose sugar structure. Tooth decay occurs when Streptococcus mutans and Streptococcus sobrinus which are germs that cause tooth decay eat the glucose or fructose in food and discharge lactic acid which in turn decays the surface of a tooth, and the germs causing tooth decay can easily break down hexose but not the xylitol which is a pentose. Therefore, acid causing tooth decay is not generated and the tooth decay germs unable to feed on nutrients separate from tooth surface. In addition, the xylitol has been used as a tooth decay prevention additive for preventing tooth decay by promoting salivation and for leaving mouth feeling cool and refreshed.

Said vegetable glycerin also known as glycerol is in a transparent syrup form with a sweet flavor. Glycerin prevents evaporation of moisture and helps supply moisture. In addition, glycerin is not harmful to the human body and has excellent moisturizing effect, and a strong moisture wicking ability. Vegetable glycerin is a natural product made from ingredients such as coconut oil and palm oil and easily dissolves in water, thereby effective in suppressing dermatitis and in particular, preventing dry mouth when used in the mouth.

Said xanthan gum is a food additive used to enhance the adherence, viscosity, and emulsion stability of food, and to enhance the properties and the texture of the food. Extracted from sugar cane, the xanthan gum is used to create gel type. In addition, the xanthan gum features thickening action, glandular capsule, and gelling effect and can also act to stabilize the adrenal glands and also for moisturizing action. Spreadability enables a good texture. Therefore, the xanthan gum can be used in food as a stabilizer, thickener, binder, emulsifiers, solidifying agent, and foaming agent.

Said Yucca recurvifolia is Monocotyledonous liliales Agavaceae evergreen shrub that is effective for rheumatism and gout as well as neuralgia, arthritis, hypertension, hyperlipidemia, and is also effective in inhibiting HIV infection, and is rich in dietary fiber and effective for bronchial asthma. The effects have been found to be contributed to the main component that is saponin (steroid hormone precursor material), saponin being the functional component contained in soybean and Korean ginseng.

In particular, the Yucca recurvifolia extract is a liquid extracted from yucca roots used as flavoring with a peculiar odor, added as a foaming agent and to preserve quality and the saponin glycosides contained therein forms bubbles when mixed with water and because of the high stability of the bubbles, user satisfaction of the toothpaste can be improved. The Yucca recurvifolia extract contained in the present invention may be procured from publicly available products on the market.

Said sodium cocoyl apple amino acid is an apple surfactant (APL, apple wash) having a very mild ingredient that doesn't irritate the skin at all, and can be used safely even on sensitive skin or baby skin. The sodium cocoyl apple amino acid is made by acylation-reacting essential amino acids obtained from apple juice and does not denature the protein of the skin and does not affect the skin cell defense system.

Said sodium phytate is used as an antioxidant and is extracted from rice bran.

Said calcium carbonate, which may be contained in the present invention, is used as an abrasive, and it is preferred that a precipitated calcium carbonate be used as an embodiment. The calcium carbonate and the precipitated calcium carbonate remove plaque attached to the tooth surface. In another embodiment, bicarbonate may be used in place of the calcium carbonate. In another embodiment of the present invention, the calcium carbonate that may be included in the toothpaste composition would preferably be made into nano-sized calcium carbonate. In this way, it is possible to reduce the wear of the tooth by using the calcium carbonate, which is a nano-sized dry raw material calcium carbonate.

Said kaolin clay of the present invention is a soft and non-abrasive clay used in medicine and cosmetics, and generates a large quantity of far infrared rays and anions as a natural mineral with the fundamental efficacy of a clay, activating the physiology of the cells generating thermal energy and thereby having photoelectric effect for discharging harmful substances, leading to excellent purification and dissolving abilities, enabling use thereof as a teeth abrasive by suctioning onto teeth and removing dental plaque.

In addition, white kaolin clay is mild and suitable for very sensitive skin, and has excellent absorption, which is very useful for attracting dirt in the pores, promoting blood circulation, soothing the skin.

Said bamboo salt, which may be included in the toothpaste composition, of the present invention is made by being baked nine times in a bamboo tube, and acts as a natural antibacterial agent. The following shows the effects of the bamboo salt.

1) Cell production: restores dead or destroyed cells rapidly, 2) anti-inflammatory: relieves inflammation, 3) blood purification: thins the blood, 4) habitus enhancement: changes the acidic habitus into alkalescent habitus to foster immunity and resistance, 5) detoxification: cleansing the toxin in the body rapidly, 6) antibacterial: germs will not form, and 7) strong reducing power enables large amount of acidic waste products to be excreted and also serves to avoid the damage caused by free radicals.

In addition, the propolis that may be contained in the toothpaste composition of the present invention is a natural antibacterial material, which is a mixture of resinic substance a bee collects from a plant such as sprout or a sap of a tree. Effects include antibacterial, antiseptic, and immunity activating actions, enhanced immune function, cell activation and cell regeneration action, anti-allergic effects, defense against genetic damage, strengthening of blood vessels, anti-oxidation, and mitigation of anticancer drug side effects. The propolis also acts to control the symptoms.

Said propolis and bamboo salt can be obtained from the publicly available products.

The present invention may comprise a natural preservative in the composition, and a preferred embodiment may comprise an anisic acid (liquid) natural antiseptic having a star anise component.

The toothpaste composition containing Curcuma longa L. according to the present invention may comprise the three essential oils, which are main components of aromatherapy recently receiving spotlight in alternative medicine, and refer to volatile organic compounds extracted from plants by distillation from plants.

In the lemon essential oil, lemon (Citrus limonum) has aldehydes and monoterpene hydrocarbons as the main components, and is known to be effective for preservation, sterilization and immune enhancement. In particular, lemons have been found to have an excellent antibacterial effect on dental decay-causing germs of oral germs.

In the spearmint essential oil, spearmint (Mentha spicata) has similar properties as peppermint but weaker in efficacy and thus safe to use on children. The oil is effective for cold, fever, influenza, asthma, bronchitis, mucosal inflammation, and sinusitis. Other uses include local anesthetics, disinfectant, anti-convulsant, astringent, carminative, cephalic agent, bile discharge accelerator, expectorant, and appetite enhancement agent.

In the peppermint essential oil, peppermint (Mentha piperita) has menthol and menthone as the main component, and is effective for treating itchiness and has antiseptic, anticonvulsant, and phlegm discharging effects.

In particular, the anticonvulsant action of peppermint is based on the effects on the nervous system, and the menthol aroma with a fresh and spicy nature improves concentration by awakening the nerves and the brain. Peppermint has an effect of a local anesthesia for muscle aches, backache, bruises, joint pain, and toothache. In addition, peppermint is one of the most effective oil for the digestive tract and effective in treating indigestion, nausea, stomach pain, diarrhea, and bloating. Peppermint contracts the blood vessels to enable faster flow of blood through the blood vessels to the spleen to promote blood cell regeneration activity. In addition, peppermint contracts and relaxes the lymph and fascia to strengthen the smooth discharge of toxins, helping to relieve headaches and migraines.

In a preferred embodiment of the toothpaste composition according to the present invention, with respect to the total toothpaste composition, 1 to 5 wt % of the Curcuma longa L. powder, 0.5 to 1.5 wt % of the natural organic sulfur, 1 to 3 wt % of the mint extract, 0.5 to 2.5 wt % of the red ginseng fermentation extract, and 7 to 10 wt % of the antibacterial combined extracts may be included.

In an embodiment of the antibacterial combined extract, with respect to the total toothpaste composition, 1 to 2 wt % of the green tea extract, 1 to 2 wt % of the dried orange peel, 2 to 3 wt % of the Houttuynia cordata Thunb. Extract, 2 to 3 wt % of the Rumex crispus extract, 1 and 2 wt % of the licorice extract, and 1 to 2 wt % of the Mori Cortex extract may be included.

An embodiment having the content of other components which may be contained in the toothpaste composition of the present invention is as shown below:

TABLE 1

| | Capacity | | Capacity |
|---|---|---|---|
| Material | (wt %) | Material | (wt %) |
| Xylitol | 25-30 | Kaolin Clay | 8-12 |
| Vegetable Glycerin | 16-20 | Bamboo salt | 0.5-1.5 |
| Xanthan gum | 0.1-1 | Propolis | 0.5-1.5 |
| Yucca recurvifolia extract | 5-7 | Anisic acid natural | 0.5-1.5 |
| | | antiseptic | |
| Sodium cocoyl apple | 4-6 | Lemon essential oil | 0.1-0.2 |
| amino acids | | | |
| Sodium phytate | 0.1-0.2 | Spearmint essential | 0.1-0.2 |
| | | oil | |
| Calcium carbonate | 12-16 | Peppermint essential | 0.05-0.15 |
| (or bicarbonate) | | oil | |

Below is a description of an embodiment that relates to the manufacturing method of a toothpaste composition containing Curcuma longa L. according to the present invention.

a) Place 18 wt % of the vegetable glycerin in container A and heat at 40 to 50° C. at medium heat, and then place 27 wt % of the xylitol therein and dissolve all, after which 0.5 wt % of the xanthan gum is placed and dissolved therein.

b) Place into container B 8 wt % of the antibacterial combined extract consisting of 1 wt % of the green tea extract, 1 wt % of the dried orange peel extract, 1 wt % of the licorice extract, 1 wt % of the Mori Cortex extract, 2 wt % of the Houttuynia cordata Thunb. extract, and 2 wt % of the Rumex crispus extract, 2 wt % of the mint extract and 2 wt % of the red ginseng fermentation extract and mix, and then add 6 wt % of the Yucca recurvifolia extract, 5 wt % of the sodium cocoyl apple amino acid and 0.1 wt % of the sodium phytate, and mix with the composition obtained in said step a).

c) Add each of the 14 wt % of the calcium carbonate, 10 wt % of the kaolin clay, 3 wt % of the Curcuma longa L. powder, 1 wt % of the natural organic sulfur and 1 wt % of the bamboo salt respectively to the mixture obtained in said step b) and then mix.

d) Add each of the 1 wt % of the propolis, 1 wt % of the anisic acid natural antiseptic having star anise as ingredient, and 0.15 wt % of the lemon essential oil, 0.1 wt % of the peppermint essential oil, 0.15 wt % of the spearmint essential oil to the composition obtained in said step c) respectively.

The final toothpaste composition obtained in said step d) can be used as toothpaste once inserted into a tube container or the like.

### EXPERIMENTATION EVALUATION OF EMBODIMENT AND COMPARATIVE EXAMPLES

Table 2 below shows a preferred embodiment of the toothpaste composition containing Curcuma longa L. in accordance with the present invention.

TABLE 2

| | Capacity | | Capacity |
|---|---|---|---|
| Material | (wt %) | Material | (wt %) |
| Xylitol | 27 | Calcium carbonate | 14 |
| | | (or bicarbonate) | |
| Vegetable glycerin | 18 | Kaolin clay | 10 |
| Xanthan gum | 0.5 | Curcuma longa L. | 3 |
| | | powder | |
| Mint extract | 2 | Bamboo salt | One |
| Red ginseng fermentation | 2 | Propolis | One |
| extract | | | |
| Antibacterial combined | 8 | Anisic natural | One |
| extract having six | | antiseptic (liquid) | |
| components | | | |
| Natural organic sulfur | One | Lemon EO | 0.15 |
| | | (Essential Oils) | |
| Yucca recurvifolia | 6 | Spearmint EO | 0.15 |
| extract (concentrate) | | (Essential Oils) | |
| Apple Wash | 5 | Peppermint EO | 0.1 |
| sodium cocoyl apple | | (Essential Oils) | |
| amino acids | | | |
| Sodium phytate | 0.1 | | |

Table 3 below shows the components of publicly available conventional toothpastes.

TABLE 3

| | Product | | |
|---|---|---|---|
| Category | name | Manufacturer | Main ingredient |
| Comparative | Perio 46 cm | LG Household | Colloidal silicon dioxide, |
| Example 1 | White Now | & Health Care | hydrogen peroxide 35% |
| | toothpaste | | |
| Comparative | 2080 | Aekyung | (Artificial coloring, |
| Example 2 | Mother's | | artificial sweeteners, |
| | Heart | | artificial flavor |
| | toothpaste | | additives) fluoride, |
| | | | vitamin E, calcium, organic |
| | | | extracts (apple), herbal |
| | | | tea mixtures (including |
| | | | natural fermented tea |
| | | | puer tea) |
| Comparative | Bamboo salt | LG Household | Dentil-type silica, bamboo |
| Example 3 | toothpaste | & Health Care | salt, Centella extract, |
| | | | glycyrrhizinate potassium, |
| | | | sodium fluoride |
| Comparative | Perio Fresh | LG Household | Glyceryl calcium |
| Example 4 | Toothpaste | & Health Care | phosphate, calcium |
| | | | carbonate, first |
| | | | sodium fluoride |

### Experimental Example 1

### Gingivitis Inhibition Experimentation

This experiment targeted 20 examinees in relatively similar environment, and all of the examinees received a first detailed oral exam before the experimentation to obtain the standard. The method used for examination was a single-blind method and all of the examinees were provided with an identical toothbrush for residual effects removal period beginning 4 weeks before the experimentation. In the experimentation, the gum was divided into buccal surface and lingual surface per one tooth to calculate the average value of the index. Each gingivitis was assessed from 0 to 3 points according to the Silness-Loe marking standard to obtain the gingival index for each tooth, after which average grade for gingival index of each entity was calculated, wherein the measurement for the experimentation was taken a total of three times in one month interval.

TABLE 4

| | Standard | After | After | After |
|---|---|---|---|---|
| Category | base | 1 month | 2 months | 3 months |
| Embodiment | 0.60 | 0.52 | 0.45 | 0.36 |
| Comparative Example 1 | 0.60 | 0.58 | 0.56 | 0.55 |
| Comparative Example 2 | 0.60 | 0.60 | 0.57 | 0.57 |
| Comparative Example 3 | 0.60 | 0.58 | 0.58 | 0.57 |
| Comparative Example 4 | 0.60 | 0.59 | 0.58 | 0.56 |

As shown in the table 4, comparative examples 1 to 4 showed a reduction of up to about 8% through and after 3 months, but the embodiment showed a reduction of 40% demonstrating to be very effective in the treatment of inflammation.

### Experimental Example 2

### Experimentation for Measuring Whitening Effects

Measurement was taken by measuring the whitening effect by having the examinees use the embodiment and the comparative examples as toothpastes for three months. The shade of the teeth was measured for the 50 selected examinees using Trubyte Bioform in advance, after which the group was divided into five groups each made up of ten people having almost identical average value of the shade (10 embodiment users, 10 users for each of the comparative examples) and each group was distributed with an embodiment and comparative examples 1 to 4 respectively to use each toothpaste composition. Among 24 types of standard criteria determined by the brightness for the Trubyte Bioform, 10 types that can be instinctively and readily identified were selected and each was given an evaluation grade for the brightness value from 1 to 10, 1 point being the lowest to 10 points being the highest. The brightness value of the teeth for each group was assessed accurate to the tenth place based on the standard before each toothpaste was used, after brushing for 3 months brightness, and the rate of increase in the average brightness value in comparison to the brightness value before the exam was calculated.

TABLE 5

| | The average | | Brightness |
|---|---|---|---|
| | brightness | The average brightness | value increase |
| Category | value (before test) | value (after 3 months) | rate (%) |
| Embodiment | 3.5 | 5.5 | 57.1% |
| Comparative | 3.5 | 3.9 | 11.4% |
| Example 1 | | | |
| Comparative | 3.6 | 4.1 | 13.8% |
| Example 2 | | | |
| Comparative | 3.5 | 4.0 | 14.2% |
| Example 3 | | | |
| Comparative | 3.7 | 4.0 | 8.1% |
| Example 4 | | | |

As shown in the table 5, the rate of increase in the brightness value for comparative examples 1 to 4 is 14.2% maximum, but the embodiment demonstrated 57.1% increase in the brightness value, confirming superior effectiveness in teeth whitening and plaque removal.

### Experimental Example 3

### Satisfaction

30 adult male and female were given the toothpaste of the embodiment and comparative examples 1 to 4 to use, and the below items were examined to display the result on the average value thereof in a table. Scores were divided from 0 (very poor) to 10 (very satisfied).

TABLE 6

| | | | | | | Cleansing | Foaming | Moisturizing | | | Overall |
|---|---|---|---|---|---|---|---|---|---|---|---|
| Category | Appearance | Color | Scent | Flavor | Viscosity | Ability | Ability | Effect | Smoothness | Freshness | Ranking |
| Embodiment | 8 | 9 | 9 | 8 | 9 | 10 | 8 | 8 | 9 | 10 | 88 |
| Comparative | 7 | 6 | 6 | 7 | 7 | 6 | 6 | 6 | 5 | 7 | 63 |
| Example 1 | | | | | | | | | | | |
| Comparative | 7 | 7 | 8 | 8 | 6 | 8 | 7 | 7 | 7 | 7 | 72 |
| Example 2 | | | | | | | | | | | |
| Comparative | 6 | 6 | 6 | 6 | 6 | 8 | 7 | 6 | 6 | 7 | 64 |
| Example 3 | | | | | | | | | | | |
| Comparative | 7 | 6 | 6 | 6 | 7 | 6 | 7 | 7 | 6 | 7 | 65 |
| Example 4 | | | | | | | | | | | |

As shown in the table 6, the toothpaste composition of the present invention as compared to the toothpaste composition components of the conventional comparative examples 1 to 4, appeared to be satisfactory in all aspects of the items, with the total score on the satisfaction confirmed to be up to 25 points or more higher.

The description of the present invention has described the specific embodiments, but it would be obvious to a person skilled in the art that a plurality of modifications are possible within limits that do not depart from the scope of the present invention.

1. A toothpaste composition containing Curcuma longa L. comprising:
Curcuma longa L. powder;
Natural organic sulfur;
Mint extract;
Red ginseng fermentation extract;
And antibacterial combined extract made by extracting at least one from the group consisting of green tea, dried orange peel, Houttuynia cordata Thunb., Rumex crispus, licorice, and Mori Cortex Radicis.

2. A toothpaste composition containing Curcuma longa L. according to claim 1, characterized by comprising 1 to 5 wt % of the Curcuma longa L. powder, 0.5 to 1.5 wt % of the natural organic sulfur, 1 to 3 wt % of the mint extract, 0.5 to 2.5 wt % of the red ginseng fermentation extract, and 7 to 10 wt % of the antibacterial combined extract, with respect to the entirety of the toothpaste composition.

3. A method for manufacturing a toothpaste composition containing Curcuma longa L. characterized by the steps of:
a) Placing and heating vegetable glycerin in container A, then placing and dissolving xylitol therein, and then placing and dissolving xanthan gum therein;
b) Placing and mixing in container B mint extract, red ginseng fermentation extract, and antibacterial combined extract made by extracting at least one from the group consisting of green tea, dried orange peel, Houttuynia cordata Thunb., Rumex crispus, licorice, and Mori Cortex Radicis, and then adding thereto Yucca recurvifolia extract, sodium cocoyl apple amino acid, and sodium phytate, and then mixing the composition obtained in said step a) therewith;
c) Adding and mixing each of calcium carbonate, kaolin clay, Curcuma longa L. powder, natural organic sulfur, and bamboo salt to the composition obtained in said step b); and
d) Adding propolis and anisic acid natural antiseptic having star anise as an ingredient, lemon essential oil, peppermint essential oil, and spearmint essential oil to the composition obtained in said step c).
